(19) United States
(12) Patent Application Publication
(10) Pub. No.: US 2011/0002998 A1
(43) Pub. Date: Jan. 6, 2011

(54) PROCESS FOR THE PRODUCTION OF INSECTICIDE-MODIFIED BEAD MATERIAL COMPOSED OF EXPANDABLE POLYSTYRENE AND INSECTICIDE-MODIFIED MOLDINGS OBTAINABLE THEREFROM

(75) Inventors: Michael Ishaque, Mannheim (DE); Olaf Kriha, Ludwigshafen (DE); Carsten Schips, Speyer (DE); Norbert Güntherberg, Speyer (DE); Daniela Longo, Siegburg (DE); Klaus Hahn, Kirchheim (DE); Bernhard Schmied, Frankenthal (DE); Clark D. Klein, Pittsboro, NC (US); Keith A. Holmes, Cary, NC (US)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/809,892

(22) PCT Filed: Dec. 5, 2008
(86) PCT No.: PCT/EP08/66844

§ 371 Date: Sep. 7, 2010

Related U.S. Application Data
(60) Provisional application No. 61/015,988, filed on Dec. 21, 2007.

(51) Int. Cl. A61K 9/14; A01N 43/36; A01N 43/54; A01N 59/14; A01N 47/22; A01N 57/16; A01N 57/14; A01N 43/88; A01N 45/02; A01N 43/56; A01N 43/40; A01N 57/30; A01P 7/04

(52) U.S. Cl. 424/486; 514/427; 514/256; 424/660; 514/479; 514/89; 514/132; 514/229.2; 514/28; 514/407; 514/326; 514/355; 514/124

(57) ABSTRACT

A process for the production of insecticide-modified bead material composed of expandable polystyrene (EPS) by extrusion, encompassing the steps of a) mixing, in a mixer, to incorporate a blowing agent and at least one insecticide from the group of the phenylpyrazoles, chlorfenapyr and hydramethylnon into a polymer melt which comprises at least one polystyrene, based on a vinylaromatic monomer, b) discharging the polymer melt comprising blowing agent, and c) pelletizing the polymer melt comprising blowing agent.

The invention relates to a process for the production of insecticide-modified bead material composed of expandable polystyrene, to insecticide-modified bead material composed of expandable polystyrene and obtainable by the process, to insecticidal moldings obtainable therefrom, to processes for their production, and also to their use in the construction industry.

Polymer foams are used by way of example in the construction industry as insulation material both above and below ground. Consumption by insects, in particular termites, can substantially damage these foams, thus compromising the insulating action, and also the mechanical stability of the moldings, and so permitting further pest encroachment. In many cases state regulations require an insecticidal protection of polymer foams because such isolation materials offer a preferred habitat for termites.

JP-2000-001564 describes the use of (±)-5-amino-1-(2,6-dichloro-α,α,α,-trifluoro-p-tolyl)-4-trifluoromethylsulfinylpyrazole (common name: fipronil) for the protection of polymer foams. Concentrations of fipronil used for this purpose are from 0.001 to 1% by weight. Polystyrene, polyethylene, and polypropylene are described as polymer matrix. The fipronil is incorporated by application to the surface of the finished molding, by application to the surface of the prefoamed foam beads, or by application to the pellets comprising blowing agent. JP 2001-259271 describes a process in which EPS pellets comprising blowing agent, or prefoamed EPS pellets, are coated with fipronil and with a binder. The processes mentioned can create undesired abraded material and dusting during production. Since this abraded material or dust comprises large amounts of active ingredient, undesired exposure is likely, as also is loss of active ingredient during production, processing, and/or use.

EP-A 0 981 956 describes the production of insecticide-modified polystyrene bead material where an insecticidal active ingredient from the group of the pyrethroids or neonicotinoids is dissolved in the monomers prior to the polymerization reaction. This type of process is not generally applicable, however, since the insecticidal active ingredients can disrupt or indeed suppress the polymerization process, preferably suspension polymerization, for example through foaming or inhibition of the polymerization reaction. Furthermore, contamination of the process water with insecticide can occur, requiring complicated treatment.

WO 00/44224 describes the production of insecticide-modified foam sheets by extrusion or molding of an expandable polymer composition which comprises, dispersed therein, an insecticide from the group of the pyrethroids. The process described relates to the production of XPS (extruded polystyrene foam), and the active ingredients used are moreover markedly different structurally from the inventive active ingredients.

It is an object of the invention to eliminate the abovementioned disadvantages and to provide a cost-effective process for the production of expandable styrene polymer pellets with long-lasting insecticidal activity.

It has been found that certain insecticidal active ingredients can be incorporated homogeneously without decomposition into a polymer melt comprising blowing agent.

The invention therefore provides a process for the production of insecticide-modified bead material composed of expandable polystyrene (EPS) by extrusion, comprising the steps of
a) mixing, in a mixer, to incorporate a blowing agent and an insecticide from the group of the phenylpyrazoles, chlorfenapyr and hydramethylnon into a polymer melt which comprises at least one polystyrene (PS), based on a vinylaromatic monomer,
b) discharging the polymer melt comprising blowing agent, and
c) pelletizing the polymer melt comprising blowing agent.

The invention further provides an EPS bead material obtainable by the inventive process, moldings obtainable from the inventive EPS bead material, a process for the production of the moldings, and also their use as a construction material, in particular insulation material, in the construction industry, in particular for the protection of buildings from termites.

EPS bead material obtainable by the inventive process comprises the active ingredient(s) preferably dispersed at the molecular level.

Dispersion of the insecticide at the molecular level in the polymer matrix gives particularly secure binding of the insecticide into the polymer matrix in the inventively produced EPS bead material. This reduces active ingredient loss and exposure to the insecticide during the production, processing, and use of the EPS bead material or of the moldings obtainable therefrom. Dispersion at the molecular level moreover permits reduction of the amount of insecticide needed.

Furthermore, the inventive moldings exhibit no disadvantages in mechanical and insulation properties when compared with a standard product (without insecticide).

For the purposes of the invention, polystyrene (PS) is used as a collective term for homo- and copolymers composed of styrene, of other vinylaromatic monomers, and, if desired, of further comonomers. PS includes by way of example standard polystyrene (general-purpose polystyrene, GPPS, usually glass-clear), impact-modified polystyrene (high-impact polystyrene, HIPS, comprising, for example, polybutadiene rubber or polyisoprene rubber), styrene-maleic acid/anhydride polymers, acrylonitrile-butadiene-styrene polymers (ABS), styrene-acrylonitrile polymers (SAN), or a mixture of these (component K1). Preferred PS is standard polystyrene, i.e. a polystyrene whose molar styrene monomer content is at least 95%.

PS also comprises blends composed of one or more of the abovementioned polymers (component K1) with one or more thermoplastic polymers (component K2), for example polyphenylene ethers (PPE), polyamides (PA), polyolefins, such as polypropylene (PP) or polyethylene (PE), polyacrylates, such as polymethyl methacrylate (PMMA), polycarbonates (PC), polyesters, such as polyethylene terephthalate (PET) or polybutylene terephthalate (PBT), polyether sulfones (PES), polyether ketones (PEK), or polyether sulfides (PES). Polyamide (PA) is a preferred thermoplastic polymer.

The polymers mentioned of component K1 are obtainable by polymerizing one or more vinylaromatic monomers, such as styrene, and, if desired, further comonomers, such as dienes, α,β-unsaturated carboxylic acids, esters (preferably alkyl esters), or amides of these carboxylic acids, and alkenes. Suitable polymerization methods are known to the skilled worker.

The vinylaromatic monomer used preferably comprises at least one compound of the general formula (I),

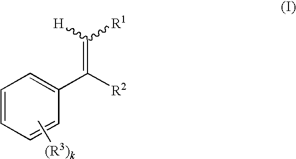

in which each of R¹ and R², independently of the other, is hydrogen, methyl, or ethyl;

R³ is hydrogen, C₁-C₁₀-alkyl, such as methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl; preferably C₁-C₄-alkyl, such as methyl, ethyl, n-propyl, isopropyl, n-butyl, isobutyl, sec-butyl and ten-butyl; and

k is a whole number from 0 to 2.

It is preferable that each of R¹ and R² is hydrogen, and it is further preferable that k=0. Particular preference is given to styrene; other particularly suitable compounds are α-methylstyrene, p-methylstyrene, ethylstyrene, tert-butylstyrene, vinylstyrene, a-vinyl-toluene, 1,2-diphenylethylene, 1,1-diphenylethylene, or a mixture of these.

Diene comonomers that can be used are any of the polymerizable dienes, in particular 1,3-butadiene, 1,3-pentadiene, 1,3-hexadiene, 2,3-dimethylbutadiene, isoprene, piperylene or a mixture of these. Preference is given to 1,3-butadiene (abbreviated to: butadiene), isoprene, or a mixture of these.

Preferred suitable α,β-unsaturated carboxylic acids or their derivatives are compounds of the general formula (II),

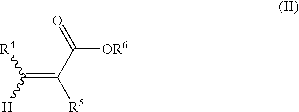

in which the definitions of the symbols are as follows:

R⁵ is selected from the group consisting of
unbranched or branched C₁-C₁₀-alkyl, such as methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl; particular preference being given to C₁-C₄-alkyl, such as methyl, ethyl, n-propyl, isopropyl, n-butyl, isobutyl, sec-butyl, and tert-butyl;
or hydrogen,
very particular preference being given to hydrogen and methyl;

R⁴ is selected from the group consisting of
unbranched or branched C₁-C₁₀-alkyl, such as methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl; particular preference being given to C₁-C₄-alkyl, such as methyl, ethyl, n-propyl, isopropyl, n-butyl, isobutyl, sec-butyl, and tert-butyl,
very particular preference being given to hydrogen;

R⁶ is selected from the group consisting of
hydrogen (whereby compound (II) is the carboxylic acid itself),
or unbranched or branched C₁-C₁₀-alkyl (whereby compound (II) is a carboxylic ester), such as methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl; particular preference being given to C₁-C₄-alkyl, such as methyl, ethyl, n-propyl, isopropyl, n-butyl, isobutyl, sec-butyl, and tert-butyl; and also 2-ethylhexyl.

Preferred compounds of the formula (II) are acrylic acid and methacrylic acid. Preference is further given to the C₁-C₁₀-alkyl esters of acrylic acid, in particular the butyl esters, preferably n-butyl acrylate, and to the C₁-C₁₀-alkyl esters of methacrylic acid, in particular methyl methacrylate (MMA).

Suitable carboxamides are in particular the amides of the abovementioned compound (II), for example acrylamide and methacrylamide.

Other monomers that can be used are compounds of the general formula (IIIa) and (IIIb), the compounds (IIIa) formally being OH-substituted carboxamides:

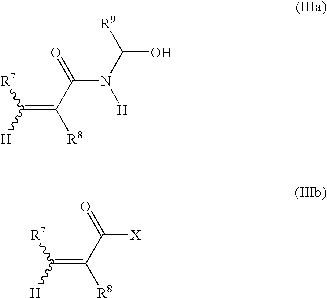

in which the definitions of the symbols are as follows:

R⁸ is selected from the group consisting of
unbranched or branched C₁-C₁₀-alkyl, such as methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl; particular preference being given to C₁-C₄-alkyl, such as methyl, ethyl, n-propyl, isopropyl, n-butyl, isobutyl, sec-butyl and tert-butyl;
or hydrogen;
very particular preference being given to hydrogen and methyl;

R⁷ is selected from the group consisting of
unbranched or branched C₁-C₁₀-alkyl, such as methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl; particular preference being given to C₁-C₄-alkyl, such as methyl, ethyl, n-propyl, isopropyl, n-butyl, isobutyl, sec-butyl and tert-butyl;
very particular preference being given to hydrogen;

R⁹ is selected from
unbranched or branched C₁-C₁₀-alkyl, such as methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl; particular preference being given to C₁-C₄-alkyl, such as methyl, ethyl, n-propyl, isopropyl, n-butyl, isobutyl, sec-butyl and tert-butyl
very particular preference being given to hydrogen;

X is selected from the group consisting of
hydrogen,
glycidyl
groups having tertiary amino groups, preferably NH(CH₂)_{b}—N(CH₃)₂, where b is a whole number in the range from 2 to 6,
enolizible groups having from 1 to 20 carbon atoms, preferably acetoacetyl, of the formula

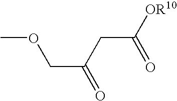

where

R¹⁰ is selected from unbranched or branched C₁-C₁₀-alkyl, such as methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl; particular preference being given to C₁-C₄-alkyl, such as methyl, ethyl, n-propyl, isopropyl, n-butyl, isobutyl, sec-butyl, and tert-butyl.

It is very particularly preferable that R⁸ in the formula (IIIa) or (IIIb) has been selected from hydrogen and methyl, and that each of R⁷ and R⁹ is hydrogen.

Methylolacrylamide is particularly preferred as compound of the formula (IIIa).

The PS can also be produced using alkenes as comonomers. Particularly suitable alkenes are ethylene (ethene) and propylene (propene).

Examples of further suitable comonomers for the production of component K1 are from 1 to 5% by weight of any of the following: (meth)acrylonitrile, (meth)acrylamide, ureido-(meth)acrylate, 2-hydroxyethyl (meth)acrylate, 3-hydroxypropyl (meth)acrylate, acrylamido-propanesulfonic acid (branched or unbranched) or the sodium salt of vinylsulfonic acid.

Suitable blowing agents are the physical blowing agents usually used in EPS bead material, examples being aliphatic hydrocarbons having from 2 to 8 carbon atoms, alcohols, ketones, ethers, or halogenated hydrocarbons, or water, or a mixture of these. It is preferable to use isobutane, n-butane, isopentane, n-pentane, or a mixture of these.

The amount used of the blowing agent is from 0.5 to 15% by weight, preferably from 1 to 10% by weight, and in particular from 2 to 8% by weight, based on the vinylaromatic monomer used.

In the inventive process, the materials added to the polymer melt used comprise not only the blowing agent but also at least one insecticide from the group of the phenylpyrazoles, in particular fipronil (IV), acetoprole, ethiprole (V), and the compound of the formula (VI), chlorfenapyr (VII), and hydramethylnon (VIII).

Particular preference is given to fipronil ((±)-5-amino-1-(2,6-dichloro-α,α,α-trifluoro-p-tolyl)-4-trifluoromethylsulfinylpyrazole), hydramethylnon, and chlorfenapyr.

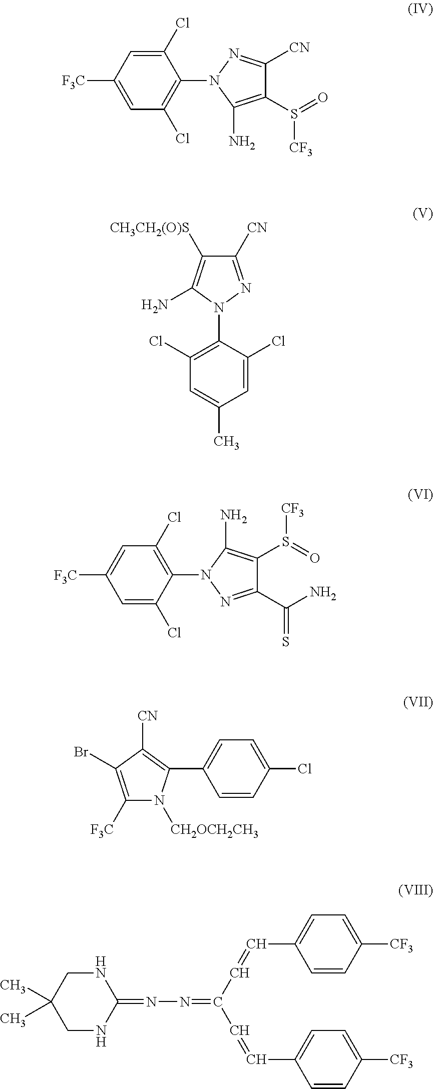

Fipronil is particularly preferred.

The compounds mentioned, in particular of the formulae (II), (III), (V), and (VI), and also their preparation, are known and described by way of example in “The Pesticide Manual”, 14th Edition, British Crop Protection Council (2006). The thiamide of the formula (IV) and its preparation is described in WO 98/28279. Fipronil, hydramethylnon, and chlorfenapyr are commercially available from BASF SE (Ludwigshafen, Germany).

The inventive EPS bead material comprises, if appropriate, (in a mixture) further insecticides, biocides, or fungicides, alongside the insecticides mentioned.

Examples of mixture constituents are those from the group of the insecticides:

I.1. Organo(thio)phosphates: acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isofenphos, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, trichlorfon;
I.2. Carbamates: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, triazamate;
I.3. Pyrethroids: allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin, profluthrin, dimefluthrin;
I.4. Growth regulators: a) chitin synthesis inhibitors: benzoylureas: chlorfluazuron, diflubenzuron, flucycloxuron, flufenoxuron, hexaflumuron, lufenuron, novaluron, noviflumuron, sulfluramid, teflubenzuron, teflumoron, buprofezin, diofenolan, hexythiazox, etoxazole, clofentazine; b) ecdysone antagonists: halofenozide, methoxyfenozide, tebufenozide, azadirachtin; c) juvenoids: pyriproxyfen, methoprene, fenoxycarb; d) lipid biosynthesis inhibitors: spirodiclofen, spiromesifen, spirotetramat;
I.5. Nicotine receptor agonist/antagonist compounds: acetamiprid, clothianidin, dinotefuran, imidacloprid, nitenpyram, thiacloprid, thiamethoxam;
I.6. GABA antagonist compounds: endosulfan, pyrafluprole, pyriprole;
I.7. Macrocyclic lactone insecticides: abamectin, emamectin, milbemectin, lepimectin, spinosad;
I.8. Site-I electron transport inhibitors:
for example, fenazaquin, fenpyroximate pyrimidifen, pyridaben, tebufenpyrad, tolfenpyrad; flufenerim, hydramethylnon, dicofol;
I.9. Site-II and site-III electron transport inhibitors:
acequinocyl, fluacyprim, rotenone;
I.10. Oxidative phosphorylation inhibitor compounds: cyhexatin, diafenthiuron, fenbutatin oxide, propargite;
I.11. Chitin biosynthesis inhibitors:
cyromazine;
I.12. Mixed function oxidase inhibitor compounds:
piperonyl butoxide;
I.13. Sodium channel modulators:
indoxacarb, metaflumizone;
I.14. Active substances with unknown or nonspecific mechanisms of action: amidoflumet, benclothiaz, bifenazate, borates, cartap, chlorantraniliprole, flonicamid, pyridalyl, pymetrozine, sulfur, thiocyclam, flubendiamide, cyenopyrafen, cyflumetofen, flupyrazofos;

The commercially available compounds of group I.1 to I.14 can be found in “The Pesticide Manual”; 14th edition, British Crop Protection Council, (2006).

Lepimectin is known from “Agro Project”, PJB Publications Ltd, November 2004. Benclothiaz and its preparation are described in EP-A1 454621. Methidathion and paraoxon and their preparation are described in “Farm Chemicals Handbook”; volume 88, Meister Publishing Company, 2001. Acetoprole and its preparation are described in WO 98/28277. Flupyrazofos is described in “Pesticide Science” 54, 1988, pages 237-243 and in U.S. Pat. No. 4,822,779. Pyrafluprole and its preparation are described in JP 2002193709 and in WO 01/00614. Pyriprole and its preparation are described in WO 98/45274 and in U.S. Pat. No. 6,335,357. Amidoflumet and its preparation are described in U.S. Pat. No. 6,221,890 and in JP 21010907. Flufenerim and its preparation are described in WO 3/007717 and in WO 03/007718. Cyflumetofen and its preparation are described in WO 04/080180.

Further preferred insecticides are amidrazones of the formula (IX),

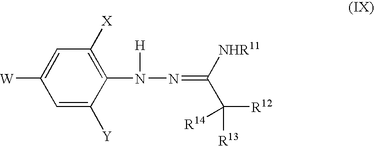

where the symbols have the following definition:
W is Cl or CF₃;
X, Y are identical or different and are Cl or Br;
R¹¹ is (C₁-C₆)-alkyl, (C₃-C₆)-alkenyl, (C₃-C₆)-alkynyl or (C₃-C₆)-cycloalkyl, which may be substituted by 1 to 3 halogen atoms, or (C₂-C₄)-alkyl which is substituted by (C₁-C₄)-alkoxy;
R¹², R¹³ are (C₁-C₆)-alkyl or, together with the carbon atom to which they are attached, form (C₃-C₆)-cycloalkyl, which may be substituted by 1 to 3 halogen atoms;
R¹⁴ is H or (C₁-C₆)-alkyl,
   and also enantiomers and salts thereof.

The symbols preferably have the following definitions in the formula (IX):
R¹¹ is preferably (C₁-C₄)-alkyl, more particularly methyl or ethyl;
R¹² and R¹³ are preferably methyl or, with the carbon atom to which they are attached, form a cyclopropyl ring which may carry one or two chlorine atoms;
R¹⁴ is preferably (C₁-C₄)-alkyl, more particularly methyl;
W is preferably CF₃;
X, Y are preferably Cl.

Further-preferred compounds of the formula (IX) are those in which X and Y are Cl, W is CF₃, R¹², R¹³ and R¹⁴ are methyl and R¹¹ is methyl or ethyl, and also those compounds in which X and Y are Cl, W is CF₃, R¹² and R¹³, together with the carbon atom to which they are attached, form a 2,2-dichlorocyclopropyl group, R¹⁴ is methyl and R¹¹ is methyl or ethyl. These compounds and their preparation are described in US 2007/0184983, for example.

Preferred mixing constituents are—alongside mixtures of the inventively used compounds with one another—pyrethroids (1.3), nicotine receptor agonists/antagonists (1.5), borate, carbaryl, chlorantraniliprole, chlorpyrifos, diflubenzuron, fenitrothion, flonicamid, flufenoxuron, hexaflumuron, indoxacarb, isofenphos, noviflumuron, metaflumizone, spinosad, sulfluramid. Particular preference is given to acetamiprid, bifenthrin, cyfluthrin, cyhalothrin, cypermethrin, alpha-cypermethrin, deltamethrin, fenvalerate, imidacloprid, lambda-cyhalothrin, permethrin, thiacloprid and thiamethoxam.

Very particular preference is given to mixtures of fipronil with one or more of the mixture constituents mentioned, in particular fipronil with α-cypermethrin. Particular preference is further given to the use of fipronil without any further mixture constituent.

The ratio of mixing between the insecticides used according to the invention and any further participants in the mixture can vary widely and is generally from 0.1:100 to 100:0.1.

Suitable concentrations of the insecticide or of the insecticide mixture, based on the EPS, are selected in such a way that the moldings obtainable therefrom have concentrations of from 10 to 1000 ppm, particularly preferably from 20 to 1000 ppm, and very particularly preferably from 50 to 500 ppm.

The EPS can comprise further additives. According to the invention, the term additives is used as a collective term for auxiliaries used during the polymerization reaction, preferably during the suspension polymerization reaction, examples being nucleating agents, plasticizers, flame retardants, IR absorbers, such as carbon black, graphite, aluminum powders, and titanium dioxide, soluble and insoluble dyes, and pigments. Preferred additives are graphite and carbon black. The preferred content of graphite is from 0.05 to 25% by weight, particularly preferred from 2 to 8% by weight, respectively based on the total weight of the EPS. The average size of the graphite particles is preferably from 1 to 50 μm, particularly preferred from 2 to 10 μm.

In one embodiment the EPS according to the invention is colored to allow a simple distinction from non-insecticide-modified EPS and, thereby, to improve product safety during production and processing.

Because of the fire-protection regulations in the construction industry and in other industries, the inventive EPS bead material preferably comprises one or more flame retardants.

An example of a suitable flame retardant is hexabromocyclododecane (HBCD), in particular the technical-grade products which essentially consist of the α-, β-, and γ-isomer, and preferably dicumyl peroxide added as synergist.

Further suitable flame retardants are for example tetrabromobisphenol-A-diallyl ether, expandable graphite, red phosphorous, triphenyl phosphate and 9,10-dihydro-9-oxa-10-phosphapenanthren-10-oxide.

To produce the inventive EPS bead material, the insecticide and the blowing agent are incorporated by mixing into a PS melt. Static or dynamic mixers, such as extruders, can be used for this mixing process. The PS melt can be directly taken from a polymerization reactor, or can be produced directly in the mixing extruder or in a separate plasticating extruder, by melting of polymer pellets. The melt can be cooled in the mixing assemblies or in separate coolers. Examples of pelletizing methods that can be used are pressurized underwater pelletization, pelletization using rotating knives and cooling by spray misting of temperature-control liquids, or spray pelletization. Examples of arrangements of apparatus suitable for conduct of the process are:

a) polymerization reactor-static mixer/cooler-pelletizer,
b) polymerization reactor-extruder-pelletizer,
c) extruder-static mixer-pelletizer,
d) extruder-pelletizer.

The arrangement can moreover have ancillary extruders for the introduction of additives, e.g. of solids or of heat-sensitive additives. The ancillary extruder can also be used to introduce the insecticide(s).

In one preferred variant, in an extruder, the insecticide is incorporated at a concentration higher than the final concentration into a polymer melt (masterbatch production), and in a second step of processing this polymer comprising active ingredient is introduced into the production process for the foamable pellets. The introduction can take place at various points of the production process for the pellets comprising blowing agent, for example through incorporation by mixing into the main stream of the polymers, shortly after the melting process, or by way of an ancillary stream, which serves to feed additives into the main stream.

In this variant, it is also possible to introduce, into the main stream, further additives which can be prepared in the form of an independent batch or together in a batch with the insecticide. Examples of possible further additives are heat stabilizers, light stabilizers, antistatic agents, flame retardants, and synergists, and also colorants and pigments.

The insecticide masterbatches and, if appropriate, also additive masterbatches can be prepared in a twin-screw extruder in compliance with the conventional safety precautions for toxic materials, for example by metering the additive powder into a melt of the carrier polymer, such as polystyrene, by way of a side-feed apparatus, and mixing with the melt by means of suitable mixing elements, such as forward-conveying and backward-conveying kneading blocks, toothed mixing elements, and toothed disks, and other screw elements which are familiar to the person skilled in the art and have mixing action.

In another variant, it is possible to mix the solid, particulate carrier polymer in the desired mixing ratio with the additives and to introduce these to a shared melting and mixing step. In order to avoid thermal degradation of the active ingredients, it is preferable to operate at temperatures which are below the conventional processing temperatures for the carrier polymers but which are high enough to give these good thermoplastic processability. Temperatures of from 150 to 210° C. are preferably selected, particularly from 160 to 200° C.

The temperature at which the polymer melt comprising blowing agent is conveyed through the die plate is generally in the range from 120 to 210° C., preferably in the range from 160 to 200° C. Cooling down to the region of the glass transition temperature is not necessary.

The die plate is heated at least to the temperature of the polymer melt comprising blowing agent. The temperature of the die plate is preferably in the range from 20 to 100° C. above the temperature of the polymer melt comprising blowing agent. This inhibits deposition derived from the polymer within the dies and ensures trouble-free pelletization.

In order to obtain marketable pellet sizes, the diameter (D) of the die perforations at the outlet of the die should be in the region from 0.2 to 1.5 mm, preferably in the region from 0.3 to 1.2 mm, particularly preferably in the region from 0.3 to 0.8 mm. This permits controlled adjustment to pellet sizes below 2 mm, in particular in the range from 0.4 to 1.4 mm, even after die swell.

Die swell can be affected not only by the molecular weight distribution but also by the geometry of the die. The die plate preferably has perforations whose L/D ratio is at least 2, where the length (L) designates that region of the die whose diameter is at most the same as the diameter (D) at the exit from the die. The L/D ratio is preferably in the range from 3 to 20.

The diameter (E) of the perforations at the entry to the die on the die plate should generally be at least twice as great as the diameter (D) at the exit from the die.

One embodiment of the die plate has perforations with conical inlet and with an inlet angle α smaller than 180°, preferably in the range from 30 to 120°. In another embodiment, the die plate has perforations with conical outlet and has an outlet angle β smaller than 90°, preferably in the range from 15 to 45°. The die plate can be equipped with perforations of different exit diameters (D) in order to give controlled production of pellet size distributions of the styrene polymers. The various embodiments of the die geometry can also be combined with one another.

A preferred process for the production of the inventive, insecticide-modified bead material composed of expandable polystyrene (EPS) by extrusion therefore encompasses the steps of
a1) mixing, in a mixer, to incorporate a blowing agent and an insecticide into a polymer melt which comprises at least one polystyrene (PS), based on a vinylaromatic monomer, in a static or dynamic mixer at a temperature of at least 150° C.,
a2) bringing the polymer melt comprising blowing agent to a temperature of at least 120° C.,
b) discharging through a die plate with perforations whose diameter at the exit from the die is at most 1.5 mm, and
c) pelletizing the polymer melt comprising blowing agent.

A particularly preferred process for the production of the inventive EPS bead material comprises the steps of
a11) polymerizing a vinylaromatic monomer and, if appropriate, further comonomers,
a12) devolatilizing the resultant polymer melt,
a13) if appropriate, mixing with further polymers,
a 14) mixing to incorporate the blowing agent, the insecticide, and, if appropriate, additives into the polymer melt in static or dynamic mixers at a temperature of from 150 to 210° C., preferably from 160 to 200° C.,
a2) if appropriate, cooling the polymer melt comprising blowing agent to a temperature which is at least 120° C., preferably from 150 to 200° C.,
c) discharging through a die plate with perforations whose diameter at the exit from the die is at most 1.5 mm, and
d) pelletizing the melt comprising blowing agent.

In step d) the pelletizing can take place directly behind the die plate underwater at a pressure in the range from 1 to 25 bar, preferably from 5 to 15 bar.

Because of the polymerization reaction in stage a11) and devolatilization in stage a12), a polymer melt is directly available for blowing-agent-impregnation in stage a14), and no melting of polymers is needed. This is not only more cost-effective but also leads to expandable polymers with low monomer contents, since the action of mechanical shear in the plasticizing region of an extruder, which generally leads to retrocleavage to give monomers, is avoided. In order to keep the monomer content low, in particular below 500 ppm, it is also advantageous to minimize the supply of mechanical and thermal energy in all of the following stages of the process. It is particularly preferable to maintain shear rates below 50/sec, preferably from 5 to 30/sec, and temperatures below 260° C., and also short residence times in the range from 1 to 20 minutes, preferably from 2 to 10 minutes, in stages c) to e). It is particularly preferable to use exclusively static mixers and static coolers in the entire process. The polymer melt can be conveyed and discharged by pressure pumps, e.g. gear pumps.

Another method for reducing the monomer content and/or level of residual solvent, such as ethylbenzene, consists in providing a high level of devolatilization by means of entrainers, such as water, nitrogen, or carbon dioxide in stage a2), or carrying out the polymerization stage a) by an anionic route. Anionic polymerization gives polymers which have not only a low monomer content but also simultaneously a very low oligomer content.

To improve processability, the finished expandable polymer pellets can be coated with glycerol esters, antistatic agents, hydrophobing agents or anticaking agents.

Suitable coating compounds are amphiphilic or hydrophobic organic compounds. Among the hydrophobic organic compounds C₁₀-C₃₀ paraffin wax, reaction products of a C₉-C₁₁ oxoalco-hol with ethylene oxide, propylene oxide or butylene oxide or polyfluoro alkyl(meth)acrylate or mixtures thereof are particularly mentioned, which can preferably be used in the form of aqueous emulsions.

Preferred hydrophobing agents are paraffin waxes with 10 to 30 C-atoms in the carbon chain, which preferably have a melting point between 10 to 70° C., particularly between 25 and 60° C. Such paraffin waxes are contained for example in the BASF-commercial products Ramasit® KGT, Persistol® E and Persistol® HP, and in Aversin® HY-N from Henkel and Cerol® ZN from Clariant.

Another group of suitable hydrophobing agents are resinlike reaction products of a N-methylol amine with a fatty acid derivative, for example a fatty acid amide, fatty amine or fatty alcohol, as described for example in U.S. Pat. No. 2,927,040 or GB-A 475 170. Their melting points are normally at 50 to 90° C. Such resins are contained for example in the BASF-commercial products Persistol® HP.

Finally polyfluoro alkyl(methyl) acrylates are also suitable, for example polyperfluoro octyl acrylate. This substance is contained in the BASF-commercial product Persistol® 0 and in Oleaphobol® from Pfersee.

Suitable amphiphilic coating compounds are antistatics, such as Emulgator K30 (mixture of secondary sodium alkane sulfonates) or glyceryl stearates, such as glyceryl monostearate or glyceryl tristearate.

According to the invention the coating of the obtained expandable styrene polymer (EPS)-granulates is carried out with water soluble, emulsifiable or suspensible coating compounds in the granulator.

A particularly preferred process for producing expandable styrene polymers comprises the steps of
a) polymerization of styrene monomers and, if appropriate, copolymerisable monomers,
b) degassing of the obtained styrene polymer melt,
c) incorporation of the blowing agent and, if appropriate, additives in the styrene polymer melt via static or dynamic mixers at temperatures of at least 150°, preferably 180 to 260° C.,
d) cooling of the blowing agent containing styrene polymer melt to temperatures of at least 120° C., preferably 150 to 200° C.,
e) if appropriate, addition of additives or fillers,
f) discharging through a die plate with perforations whose diameter at the exit from the die is at most 1.5 mm, and
g) pelletizing the melt containing the blowing agent directly behind the die plate under water, in the presence of a solution, emulsion or suspension of the coating compound.

Variable back pressure in an under water granulator (UWG) gives the possibility to specifically produce compact or partially prefoamed granulates. In common the pelletizing (granulation) is carried out at pressures in the range of 1 to 25 bar, preferred 5 to 15 bar. If a nucleation agent is used, the partial prefoaming at the die of the UWG keeps controllable.

The EPS-granulates are coated with commonly 0.1 to 0.5% by weight, preferably 0.1 to 0.3% by weight, respectively based on the solid content of the coating compound. The applied amount of coating compound can be adjusted e.g. by the concentration in the water cycle. The coating compound is used in the water cycle of the under water granulator commonly in amounts in the range of 0.05 to 20% by weight, preferably in the range of 0.1 to 10% by weight, based on the solid content in the water. For constant coating quality the concentrations of the coating compound in the water cycle should be kept constant, e.g. via constant dosing of the coating compound according to the discharge via the coated EPS.

The temperature of the water cycle in the UWG should be below the glass transition temperature of the styrene polymer, so that the EPS-granulate is coated only on the surface and no complete impregnation takes place. Preferably the temperature is in the range of 5 to 80° C., particularly preferred in the range of 10 to 60° C.

The invention moreover provides insecticide-modified bead material composed of expandable polystyrene (EPS) and obtainable by the inventive process, where this comprises at least one insecticide from the group of the phenylpyrazoles, chlorfenapyr, and hydramethylnon, preferably dispersed at the molecular level in the PS matrix.

Dispersion of the insecticide at the molecular level in the polymer matrix, in the inventive insecticide-modified EPS bead material gives firm binding of the insecticide into the polymer matrix. There is also corresponding binding of the insecticide in the polymer matrix of the moldings obtainable from the insecticide-modified EPS bead material. This reduces loss of active ingredient and exposure to the insecticide during the production, processing, and use of the EPS bead material or of the moldings obtainable therefrom. Dispersion at the molecular level also permits reduction of the amount of insecticide needed.

According to the invention, dispersion at the molecular level means that the dispersion of the active ingredient in the polymer matrix is so fine that no crystalline content of the active ingredient can be detected by X-ray diffractometry. The term “solid solution” is also used for this type of condition.

Since the detection limit for crystalline content in X-ray diffractometry is about 3% by weight, the expression “no crystalline content” means that less than 3% by weight of crystalline content is present. The differential scanning calorimetry (DSC) method can be used to determine the condition of dispersion at the molecular level. In the case of dispersion at the molecular level, there is no remaining melting peak observable in the region of the melting point of the active ingredient. The detection limit of this method is about 1% by weight.

An important demand placed upon solid solutions is that they are stable even when stored for a prolonged period, i.e. that the active ingredient does not crystallize out. Another important factor is solid solution capacity, in other words the ability to form stable solid solutions with maximum active ingredient contents.

For the production of molding from the inventive EPS bead material, this can be used in pure form or in a mixture with further EPS bead material, in particular insecticide-free EPS bead material.

The mixing ratio of inventive and further EPS bead material here can be varied freely, but is preferably selected in such a way that the moldings produced have concentrations of from 10 to 1000 ppm, particularly preferably from 20 to 1000 ppm, and very particularly preferably from 50 to 500 ppm.

Mixing ratios of inventive EPS bead material to further EPS bead material are preferably from 1000:1 to 1:1000, particularly preferably from 100:1 to 1:100, very particularly preferably from 50:1 to 1:50, and in particular from 10:1 to 1:10.

The invention also provides a process for the production of inventive moldings. For this, known methods familiar to the person skilled in the art are preferably used, in a first step d) partially prefoaming EPS bead material obtainable or obtained according to the invention, preferably by means of hot air or steam, and in a second step e) fusing it to give moldings. The fusion can be brought about by foaming-to-completion (foaming-to-completion process) (ea) or press molding (press molding process) (eb).

In the foaming-to-completion process (ea), which is preferred, in step (ea1) the inventive prefoamed EPS bead material is charged to a gastight mold. The term gastight is not intended to exclude the possibility that small amounts, for example up to 10% by volume, of the gas volume present in the mold or of the gas volume produced during the process of foaming to completion escape from the mold.

The geometry (three-dimensional shape) of the gastight mold usually corresponds to the desired geometry of the subsequent molding. For foam sheets, for example, a simple box-shaped mold is suitable. In the case of more complicated shapes, it can be necessary to compact the bed of bead material charged to the mold, as described for the press molding process.

Since the beads are intended to fuse to one another during the subsequent foaming-to-completion process, it is advantageous to fill the mold up to its brim with the beads, so as to minimize the unfilled volume in the mold.

In step (ea2), the charge of bead material in the closed mold is foamed to completion by controlling the temperature of the material to from 60 to 120° C., preferably from 70 to 110° C. (for example using steam or any other heat-transfer medium). The beads fuse here to give the molding, in that the interstices in the loose bead material are filled by the expanding beads, and the softened beads “fuse” with one another.

The pressure during the foaming-to-completion process is not usually critical, and is generally from 0.05 to 2 bar. The duration of the foaming-to-completion process depends inter alia on the size and shape of the molding and also on its desired density, and can vary widely.

In step (ea3) of the foaming-to-completion process, the resultant molding is removed from the mold, and this can take place manually or automatically by means of conventional ejector apparatuses or demolding apparatuses.

The inventive process for the production of the moldings where fusion is undertaken by foaming-to-completion (ea) therefore encompasses the steps of:
(ea1) charging the prefoamed EPS bead material according to the invention to a gastight mold,
(ea2) in the closed mold, fusing the charge of bead material by controlling its temperature to from 60 to 120° C., whereupon, by foaming, the bead material fuses to give a molding, and
(ea3) removing the resultant molding.

The density of the moldings obtained by this foaming-to-completion process is usually from 10 to 100 g/l, preferably from 15 to 80 g/l, and particularly preferably from 15 to 60 g/l, determined to DIN 53420. The moldings preferably do not have any pronounced density gradient, i.e. the density of the peripheral layers is not markedly higher than that of the inner regions of the molding.

In the press molding process, in step (eb1), the inventive prefoamed EPS bead material is charged to a gas-permeable mold. The gas permeability can, for example, be achieved through holes which are provided in the mold and which are preferably such that they are not blocked by the polymer (see below) during the subsequent press molding process step (eb2), for example because they are of low diameter.

The shape (three-dimensional shape) of the gas-permeable mold generally corresponds to the desired shape of the subsequent molding. If the intention is to produce foam sheets, a simple box-shaped mold can be used. In particular for complicated shapes, it can be necessary to compact the bed of bead material charged to the mold and thus eliminate undesired cavities. Examples of methods for compaction here are shaking of the mold, tumbling movements, or other suitable measures.

Since the bead material is then pressed, it is not—unlike the process described at a later stage below for foaming-to-completion—preferable, but nor is it disadvantageous, that the mold be filled to its brim with the bead material. The fill level depends inter alia on the desired thickness of the subsequent molding.

In step (eb2), the charge of bead material is pressed to give a molding, with volume reduction. The volume reduction is generally from 1 to 80% by volume, preferably from 5 to 60% by volume, and in particular from 10 to 50% by volume, based on the volume of the charge of bead material prior to the press molding process.

The temperature during the press molding process is usually from 20 to 100° C., preferably from 30 to 90° C., and in particular from 40 to 80° C. Examples of the method of temperature control are electrical heating or heat-transfer media. The pressure maximum during the compression procedure, or the locking force of the press, and also the duration of the press molding process (press time) depend inter alia on the size and shape of the molding, and also on its desired density, and can be varied widely.

The gas-permeability of the mold ensures that blowing agent present in and between the beads, air, or other gases can escape uniformly during the press molding process. To the extent that the coated bead material has been used in a form which has not been dried but has a “moist” coating, the volatile auxiliaries also escape, an example being the water comprised in the coating composition.

Conventional presses using press mold and ram, for example multi-daylight presses, are suitable for the press molding process. The temperature of the mold or of the ram, or of both components, can be controlled here.

In step (eb3), the molding obtained in step (eb2) is hardened, by controlling its temperature, or that of the mold, to from 20 to 100° C., preferably from 30 to 90° C., and in particular from 40 to 80° C. The temperature-control method can, for example, be electrical heating or use of heat-transfer media. The pressure during the hardening process, and the hardening time, depend inter alia on the size and shape of the molding, and vary widely.

The temperatures and pressures mentioned do not have to be maintained during the entire hardening time; instead, it is also possible to allow the molding to stand, for example at room temperature and ambient pressure, for a certain time, during which it hardens completely. The hardening process can take place with the mold closed or opened.

The temperature, pressure, press time, and other conditions during the press molding process can be selected within the ranges mentioned for step (eb2) in such a way that the molding hardens before the press molding process has finished, i.e. the press-molding step (eb2) and the hardening step (eb3) become combined. As an alternative, step (eb3) can be executed following step (eb2), and the conditions mentioned for step (eb3) then apply here.

In step (eb4) of the press molding process, the resultant molding is removed from the mold. This can take place manually or automatically by means of suitable ejector apparatuses or demolding apparatuses.

The inventive process for the production of a molding where fusion e) is undertaken by press molding therefore encompasses the steps of:
(eb1) charging the bead material according to the invention to a gas-permeable mold,
(eb2) in the closed mold, press molding of the charge of bead material, with volume reduction, to give a molding,
(eb3) hardening of the molding by controlling temperature to from 20 to 100° C., and
(eb4) removing the resultant molding.

The density of the moldings obtained by this press molding process is generally from 15 to 120 g/l, preferably from 20 to 100 g/l, and particularly preferably from 20 to 70 g/l to, DIN 53420. The moldings preferably do not have any pronounced density gradient, i.e. the density of the peripheral layers is not markedly higher than that of the inner regions of the molding.

Further information concerning conventional impregnation and foaming processes is found by way of example in Kunststoffhandbuch [Plastics Handbook], volume 5, Polystyrol [Polystyrene], edited by R. Vieweg and G. Daumiller, Carl Hanser Verlag Munich 1969.

It is preferable to begin by producing blocks as molding and then to divide the blocks to give sheets, for example by cutting or sawing.

The inventively prefoamed EPS bead material can be processed not only to give blocks but also to give moldings of any type. The moldings are preferably semifinished products (sheets, pipes, rods, profiles, etc.) or other moldings of simple or complex design. The moldings are preferably sheets, in particular foam sheets.

The thickness of the foam sheets can vary widely and is usually from 1 to 500, preferably from 10 to 300 mm. The length and width of the sheets can likewise be varied widely. It is limited inter alia by the size of the mold (compression mold or foam mold) and, in the case of the press molding process, by the locking force of the press used.

The invention further provides moldings obtainable from the inventive EPS bead material, which comprise, dispersed at the molecular level in the polymer matrix, an insecticide from the group of the phenylpyrazoles, chlorfenapyr, and hydramethylnon.

Preference is given to the use of the inventively produced moldings in the construction industry, for example as insulation material either below or above the ground, for avoidance or mitigation of the damage caused to these moldings by pests, for example insects, consumption by which can bring about substantial damage to the moldings, thus compromising the insulating action of the moldings and also their mechanical stability, and permitting further encroachment of the pests.

The inventive moldings can be used advantageously in moldings which are constantly exposed to water, for example for plates for roof isolation or perimeter insulation, for floating bodies or water sensitive packaging materials as boxes for fish.

The inventively produced moldings are particularly suitable for the avoidance or mitigation of damage by termites.

The invention, therefore, additionally provides the use of the inventive moldings for the protection of buildings from termites, and a process for the protection of a building from termites, where the inventive moldings are incorporated into the base, the outwalls or the roof of the building to be protected.

The invention is further illustrated by the examples, but not restricted thereby.

### EXAMPLES

### 1. Starting Materials

### a) EPS

The polystyrene melts comprising blowing agent and used for the examples were composed of PS 158 K from BASF Aktiengesellschaft with viscosity number VN of 98 ml/g (M_{w}=280 000 g/mol, polydispersity Mw/Mn=3.0) and 6% by weight of n-pentane.

### b) Insecticide Component

The insecticide component (A) used comprised an aqueous suspension concentrate of fipronil (Thermidor®SC), which comprises 9.1% of fipronil and is available from BASF SE.

### Example 1

### Production of a Fipronil-Masterbatch-Granulate

A twin-screw kneader with a screw diameter of 30 mm and with a processing length of 24 D, equipped with a feed zone, a transition zone, a mixing zone with forward-conveying and non-conveying kneading blocks, and a metering zone, terminated by a die plate having 2 perforations of diameter 3 mm was supplied by way of a weigh feeder with a homogeneous mixture composed of 99.0% by weight and 1.0% by weight of fipronil. The screw rotation rate was 300 rpm and the temperature was from 180 to 200° C. The mixture was run through the extruder with 10 kg/h throughput and, after exit from the dies, cooled in a water bath and pelletized. The pellets thus obtained were used in example 2.

### Example 2

### Production of Insecticidal Moldings by Using the Masterbatch from Example 1

The polystyrene melt comprising blowing agent and comprising fipronil (6% by weight of n-pentane, various proportions of fipronil) was extruded at 100 kg/h throughput through a die plate having 300 perforations (diameter at exit from die (D) 0.4 mm).

The melt temperature was 160° C. The pellet diameter of the expandable polystyrene pellets obtained, comprising fipronil, was uniform, at 1.0 mm.

The pellets were coated with 0.3% of glycerol monostearate and prefoamed using a current of steam in a commercially available prefoamer to a bulk density of 15 g/l. After an intermediate storage time of 24 hours, they were fused in a gastight block mold, using a current of steam.

The foam blocks were then cut to give foam sheets and the proportion of fipronil was determined.

The proportion of fipronil in the samples thus prepared is shown in Table 1.

TABLE 1

| | Polystyrene portion added | Proportion of fipronil |
|---|---|---|
| Sample | from Example 1 [%] | in the foam [ppm] |
| 1 | 0.3 | 28 |
| 2 | 0.5 | 37 |
| 3 | 1.0 | 98 |
| 4 | 5.0 | 487 |
| 5 (reference) | 0 | 0 |

The following method was used to determine insecticidal activity:

### Example 3

### Biological Tests to Determine the Activity of Foam Blocks Comprising Fipronil from Example 2

The bioassay method was similar to the soil termiticide bioassay method described in Su et al. (1993). Out of foamblocks according to the invention cylinders (ca. 2.5-cm diameter by 5.0-cm length) were cut using a 2.5-cm diameter plug cutter and drill press. Each polystyrene cylinder was wedged into a 2.5-cm diameter Tenite® butyrate tube. This tube was then connected by a Tygon tubing collar to another tube containing 80 workers and one soldier. The 5.0-cm polystyrene cylinder was sandwiched between two 3-cm agar segments. Pieces of southern yellow pine and paper strips provided food and harborage for termites in both the tube with termites and the tube with the polystyrene cylinder so that the termites had a source of food both above and below the polystyrene cylinder. The tubes were held at 25° C. during the 7 d of the bioassay.

The distance tunneled through the outside surface of cylinders along the interior wall of the tube was recorded every 24 h. Short (<10 mm) straight tunnels on the exterior of cylinders were measured with a ruler. Longer, curved tunnels were measured by placing a section of a thin rubber band along the length of the tunnel and then measuring the length of the rubber band. The bioassay was terminated after 7 d. At termination, mortality as well as distance tunneled through the interior of cylinders was determined. Tunnels on the interior of cylinders were measured by threading a small piece of 24 gauge insulated telephone wire through a tunnel, withdrawing the wire, and measuring its length with a ruler. To estimate the amount of tunneling through the interior of cylinders that occurred each day, the total distance tunneled through cylinder exteriors during the 7-d bioassay was proportion by day, then the amount of interior tunneling was adjusted according to the proportion of total tunneling that occurred each day.

The results of the biological tests are shown in Table 2.

TABLE 2

| | | Mean | Mean | Mean |
|---|---|---|---|---|
| | Mean | external | internal | total |
| | mortality | tunneling | tunneling | tunneling |
| Sample | [%] | [cm] | [cm] | [cm] |
| 1 | 98.5 | 7.9 | 0.9 | 8.8 |
| 2 | 96.3 | 7.1 | 3.8 | 10.8 |
| 3 | 82.7 | 8.4 | 5.1 | 13.5 |
| 4 | 92.6 | 3.8 | 4.9 | 8.7 |
| 5 (Reference | 13.0 | 10.7 | 6.9 | 17.6 |
| without | | | | |
| fipronil) | | | | |

### Example 4

### Production of Insecticidal Moldings Using Termidor® SC

Mixtures composed of polystyrene 158 K (BASF) and fipronil (in the form of Thermidor® SC) were processed by minipelletization to give pellets of diameter about 1 mm and length from about 1 to 1.5 mm. The constitutions of the minipellets obtained were as follows:

| | Polystyrene | Fipronil |
|---|---|---|
| Sample | content [%] | content [%] |
| 6 | 99.95 | 0.05 |
| 7 | 99.98 | 0.02 |
| 8 | 99.99 | 0.01 |
| 9 | 99.995 | 0.005 |

The minipellets (4 kg) of samples 1 to 4 were used as initial charge together with 4.00 kg of precipitated magnesium pyrophosphate and 0.50 kg of polyvinylpyrrolidone (Luvitec® K30 1% strength, BASF SE), in 40.00 kg of demineralized water at 130° C. 0.28 kg of pentane were metered into the material within a period of one hour and stirring was continued for 10 hours at 130° C. The reaction mixture was cooled to room temperature and discharged by way of a sieve. The product obtained was dried overnight at room temperature on a sieve of dimensions about 1.5 m² and then processed to give foam sheets.

1.-15. (canceled)

16. A process for the production of insecticide-modified bead material composed of expandable polystyrene (EPS) by extrusion, encompassing the steps of
a) mixing, in a mixer, to incorporate a blowing agent and at least one insecticide from the group of the phenylpyrazoles, chlorfenapyr and hydramethylnon into a polymer melt which comprises at least one polystyrene, based on a vinylaromatic monomer,
b) discharging the polymer melt comprising blowing agent, and
c) pelletizing the polymer melt comprising blowing agent.

17. The process according to claim 16, wherein the insecticide is fipronil.

18. The process according to claim 16, which comprises at least one further insecticide contained in the polymer melt.

19. The process according to claim 18, wherein the further insecticide is selected from the group consisting of pyrethroids, nicotine receptor agonist/antagonist compounds, borates, carbaryl, chlorantraniliprole, chlorpyrifos, diflubenzuron, fenitrothion, flonicamide, flutenoxuron, hexaflumuron, indoxacarb, isofenphos, noviflumuron, metaflumizone, spinosad and sulflur amide.

20. The process according to claim 16, wherein the concentration of the insecticide in the EPS bead material is from 10 to 1000 ppm.

21. The process according to claim 19, wherein the concentration of the insecticide mixture in the EPS bead material is from 10 to 1000 ppm.

22. The process according to claim 20, wherein the concentration of the insecticide in the EPS bead material is from 50 to 500 ppm.

23. The process according to claim 21, wherein the concentration of the insecticide mixture in the EPS bead material is from 50 to 500 ppm.

24. An EPS bead material obtainable by the process according to claim 16.

25. The EPS bead material according to claim 24, wherein the insecticide has been dispersed at the molecular level in the polymer matrix.

26. A process for the production of moldings composed of the EPS bead material according to claim 24, wherein the EPS bead material
d) is partially prefoamed, and then e) fused to give moldings.

27. The process according to claim 26, wherein the EPS bead material according to is mixed with further EPS bead material.

28. A molding, obtainable from the EPS bead material according to claim 24.

29. A molding obtainable by the process according to claim 26.

30. An insulation material which comprises the moldings according to claim 28.

31. A process for the protection of buildings from termites which comprises utilizing the molding material as claimed in claim 29.

32. A process for the protection of a building from termites which comprises assembling the moldings according to claim 28 in the base, the outwalls or the roof of the building to be protected.
